(19) United States
(12) Patent Application Publication
(10) Pub. No.: US 2025/0291823 A1
(43) Pub. Date: Sep. 18, 2025

(54) INFORMATION PROCESSING APPARATUS, INFORMATION PROCESSING METHOD, AND NON-TRANSITORY COMPUTER READABLE MEDIUM

(71) Applicant: KABUSHIKI KAISHA TOSHIBA, Tokyo (JP)

(72) Inventors: Sunao YOTSUTSUJI, Kawasaki Kanagawa (JP); Toru YANO, Shinagawa Tokyo (JP); Myungsook KO, Ota Tokyo (JP); Masaaki TAKADA, Kashiwa Chiba (JP); Gen LI, Kawasaki Kanagawa (JP)

(73) Assignee: KABUSHIKI KAISHA TOSHIBA, Tokyo (JP)

(21) Appl. No.: 19/062,136

(22) Filed: Feb. 25, 2025

(30) Foreign Application Priority Data
Mar. 12, 2024 (JP) 2024-038430

(51) Int. Cl. G06F 16/3329; G16B 50/30

(52) U.S. Cl. CPC G06F 16/33295; G16B 50/30

(57) ABSTRACT

According to one embodiment, an information processing apparatus comprising processing circuitry configured to: analyze data regarding genetic information according to a purpose of a data analysis and obtain analysis result data; and generate a prompt for inputting into a language model, the prompt instructing to interpret the analysis result data based on gene-related information related to the analysis result data and the purpose of the data analysis and obtain an interpretation result of the analysis result data based on the prompt and the language model.

### CROSS REFERENCE TO RELATED APPLICATIONS

This application is based upon and claims the benefit of priority from the prior Japanese Patent Application No. 2024-038430, filed on Mar. 12, 2024, the entire contents of which are incorporated herein by reference.

### FIELD

The present embodiments relate to an information processing apparatus, an information processing method, and a non-transitory computer readable medium.

### BACKGROUND

An integrated analysis on plural datasets related to genetic information is a useful technique for analyses with regard to diseases and exploration of biomarkers. Such an integrated analysis is called a meta-analysis. Since heredity information is biologically derived measured values, a single dataset has a relatively small number of samples, and the integrated analysis on plural datasets is required in many cases. In a field of genetic information, information processing apparatuses and methods specialized in the meta-analysis have been developed.

However, in order to use the information processing apparatuses and methods specialized in the meta-analysis, advanced expertise in genetic information has been required for searching and selecting datasets and applying an analytical technique. In addition, biological knowledge and statistical knowledge have been required to interpret results of the meta-analysis. In this way, conducting the meta-analysis requires a lot of effort and time, which is a high barrier for non-experts. Therefore, it is desirable to conduct a meta-analysis without requiring advanced expertise in genetic information, enabling the analysis in a short period.

### BRIEF DESCRIPTION OF THE DRAWINGS

FIG. 1 is a block diagram showing a configuration example of an information processing apparatus according to an embodiment of the present invention.

FIG. 2 is a block diagram showing a configuration example of a dataset searcher according to the embodiment of the present invention.

FIGS. 3A and 3B show examples of selected datasets and evaluation results thereof, along with selection information of the selected datasets according to the embodiment of the present invention.

FIG. 4 is a block diagram showing a configuration example of a meta-analyzer according to the embodiment of the present invention.

FIGS. 5A and 5B show examples of analysis results and processed analysis results according to the embodiment of the present invention.

FIG. 6 is a block diagram showing a configuration example of an interpretation generator according to the embodiment of the present invention.

FIG. 7 shows a template for an interpretation generation prompt according to the embodiment of the present invention.

FIG. 8 shows an example of the interpretation generation prompt according to the embodiment of the present invention.

FIG. 9 shows an example of interpretive information according to the embodiment of the present invention.

FIG. 10 shows an example of user presentation data presented to a user.

FIG. 11 is a flowchart schematically showing a procedure in the information processing apparatus according to the embodiment of the present invention.

FIG. 12 is a flowchart showing an example of a procedure for generating interpretive information for analysis results according to the embodiment of the present invention.

FIG. 13 is a hardware block diagram of the information processing apparatus according to the embodiment of the present invention.

### DETAILED DESCRIPTION

According to one embodiment, an information processing apparatus comprising processing circuitry configured to: analyze data regarding genetic information according to a purpose of a data analysis and obtain analysis result data; and generate a prompt for inputting into a language model, the prompt instructing to interpret the analysis result data based on gene-related information related to the analysis result data and the purpose of the data analysis and obtain an interpretation result of the analysis result data based on the prompt and the language model.

Hereinafter, embodiments of the present invention will be described with reference to the drawings.

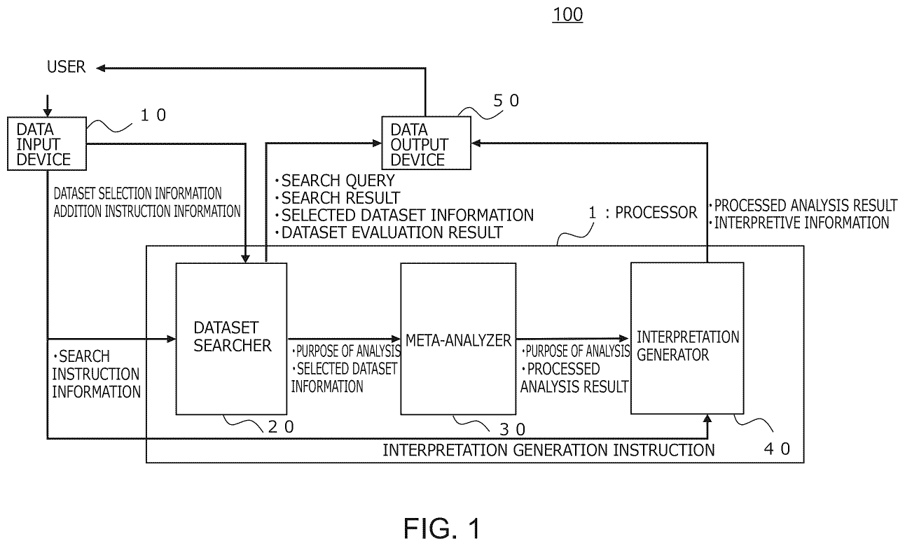

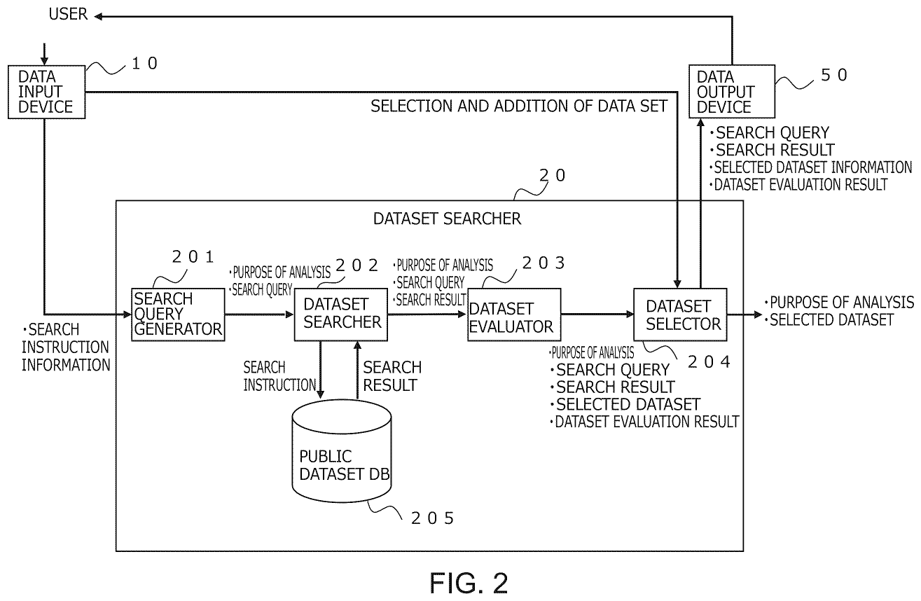

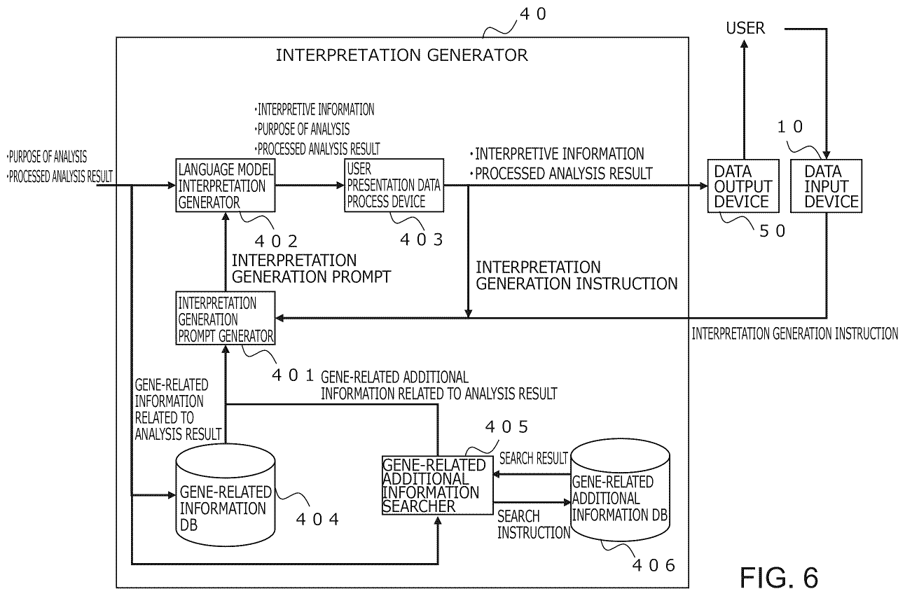

FIG. 1 is a block diagram schematically showing a configuration of an information processing apparatus 100 according to an embodiment of the present invention. The information processing apparatus 100 according to the present embodiment includes a processor or processing circuitry 1, a data input device 10 (input device), and a data output device 50 (output device). The processor 1 includes a dataset searcher 20, a meta-analyzer 30, and an interpretation generator 40. The dataset searcher 20 includes, as a database (DB), a public dataset DB 205 which is a third database (see FIG. 2), and the interpretation generator 40 includes, as DBs, a gene-related information DB 404 which is a first database and a gene-related additional information DB 406 which is a second database (see FIG. 6).

The data input device 10 receives various instructions or information from a user as inputs. For example, as instructions or information for the dataset searcher 20, search instruction information for the dataset is accepted. The search instruction information includes both or at least one of information on a purpose of an analysis and search condition information. In addition, the data input device 10 receives information (dataset selection information) for specifying a dataset to be selected from plural datasets obtained by the dataset searcher 20 or information (dataset addition instruction information) for specifying addition of a dataset which is not obtained by the dataset searcher 20. The data input device 10 may also receive information (interpretation generation instruction information) indicating an instruction for generating an interpretation to the interpretation generator 40 from the user.

A method and a format by which the data input device 10 receives these information may include input in a text format where the user types characters using a keyboard, input where the user clicks on an option or a button on a screen using a mouse, input where the user taps on an option or a button on the screen using a touch panel, input where the user inputs characters using voice with a microphone, input where the user inputs characters using facial or hand movements with a camera, input where the user writes characters by hand using a pen, and the like.

As an illustrative example for the description of the present embodiment, there is a sentence stating, “it is desired to explore microRNA biomarkers that are effective for distinguishing pancreatic cancer” as information on the purpose of the analysis. As for the search condition information, in this example, there is information indicating a condition of using a gene expression omnibus (GEO) database, and information indicating a condition that measuring instrument is limited to microarrays.

The dataset searcher 20 generates a search query based on the search instruction information obtained from the data input device 10, uses the search query to search the public dataset DB 205, and retrieves plural datasets. For example, if the search condition information includes a condition that a specific database is to be used, a search is conducted in a database that meets the condition. If the search condition information includes a condition that measuring instrument is to be specified, a dataset measured by the measuring instrument is set as a target of the search. The dataset searcher 20 evaluates the obtained plural datasets, selects two or more databases, and outputs the datasets that have been selected (selected datasets). In the present embodiment, two or more databases are selected, but depending on a purpose of a data analysis, selecting a single database is not excluded.

FIG. 2 is a block diagram showing a configuration example of the dataset searcher 20. The dataset searcher 20 includes a search query generator 201, a dataset searcher 202, a dataset evaluator 203, a dataset selector 204, and the public dataset DB 205.

The search query generator 201 generates a search query based on search instruction information. More specifically, a search query is generated based on at least one of information on a purpose of an analysis and search condition information which are included in the search instruction information.

The search query is a keyword or a query to be used to search the public dataset DB 205. Herein, a text of the obtained information on the purpose of the analysis may be used as the search query as is, or the text of the obtained information on the purpose of the analysis to which the text of the search condition information is added to may be used as the search query. A search query may be generated by processing information using a rule-based method, or a search query may be generated by processing information using a statistical technique or a machine learning technique.

In the aforementioned example, keywords related to genetic information, “pancreatic cancer” and “microRNA”, are extracted from a sentence “it is desired to explore microRNA biomarkers that are effective in distinguishing pancreatic cancer”. A phrase “pancreatic cancer AND microRNA AND microarray” is generated as the search query so as to include the extracted keywords and information of the measuring instrument and to conform to a GEO database search query.

The public dataset DB 205 stores or accumulates publicly available datasets (public datasets). For example, the public dataset includes an identification ID of the public dataset, a title, an overview, the number of samples, a measurer, a measurement target, measuring equipment, a measurement value, a related research, and the like.

The dataset searcher 202 uses the search query generated by the search query generator 201 to instruct a search in the public dataset DB 205 to retrieve plural searched datasets. In the aforementioned example, by using the search query “pancreatic cancer AND microRNA AND microarray”, the dataset searcher 202 searches the GEO database, which is the public dataset DB 205, to retrieve plural datasets as search results.

The dataset evaluator 203 evaluates plural datasets retrieved through the search. An evaluation of the dataset may be performed based on attribute information of the dataset or performed based on the measurement data itself. Evaluation criteria may relate to a quality, a reliability, and data availability related to the meta-analysis or to a relevance of information on the purpose of the analysis and the search condition information. The evaluation criteria may vary depending on the purpose of the meta-analysis and the information of the search condition.

In the aforementioned example, the dataset evaluator 203 extracts the measurement target genes of the measuring instrument and the number of measured samples which are included in the datasets as an evaluation of plural datasets and calculates the number of overlaps of the measurement target genes among the plural datasets. It is also checked if the same dataset (duplicate dataset) exists among the plural datasets. The number of measured samples may also be checked in each dataset.

The dataset selector 204 determines selection criteria according to the evaluation criteria mentioned above and selects those datasets that meet the selection criteria from among the plural datasets evaluated by the dataset evaluator 203. Information of the dataset that has been selected (selected dataset) is output to the user through the data output device 50. The information of the dataset to be output to the user includes, for example, a dataset name and information extracted or calculated for the evaluation in the dataset evaluator 203. The information in the dataset output to the user may or may not include the measurement data itself.

The dataset selector 204 may obtain at least one of information (dataset selection information) indicating plural datasets selected by the user from among the selected datasets and information (dataset addition information) on a dataset to be added from the data input device 10. If the dataset selection information has been obtained, a set of selected datasets is updated by selecting two or more datasets from the selected datasets (by removing the datasets that have not been selected). If the dataset addition information has been obtained, the set of selected datasets is updated by adding the dataset indicated by the dataset addition information to the selected datasets. The dataset indicated by the dataset addition information may be directly provided by the user or obtained from the public dataset DB 205. If the dataset selector 204 does not receive either the dataset selection information or the dataset addition information from the user, the selected datasets selected by the dataset selector 204 itself are adopted as is.

The dataset selector 204 may adopt the selected datasets as is without outputting the selected datasets to the user. In this way, the processing related to the selection of the dataset may be executed solely by the dataset selector 204 based on an algorithm provided in the dataset selector 204, or part or all of the processing may be carried out based on a user's decision.

In the aforementioned example, for instance, the dataset selector 204 eliminates duplicates of the datasets and selects datasets where the number of measured samples is greater than or equal to a threshold (removing datasets with the number of measured samples below the threshold). In addition, plural datasets (two or more) are selected so that the number of overlaps of the measurement target genes becomes greater than or equal to the threshold.

The dataset selector 204 automatically eliminates duplicates of the datasets and selects datasets with the number of measured samples greater than or equal to the threshold (removing datasets with the number of measured samples below the threshold), and outputs the resulting dataset to the user. The user may select two or more datasets from among these output datasets to ensure that the number of overlaps of the measurement target genes does not fall below the threshold and input the above dataset selection information indicating the selected datasets. In this way, part or all of the processing related to the selection of the dataset may be carried out based on the user's instructions.

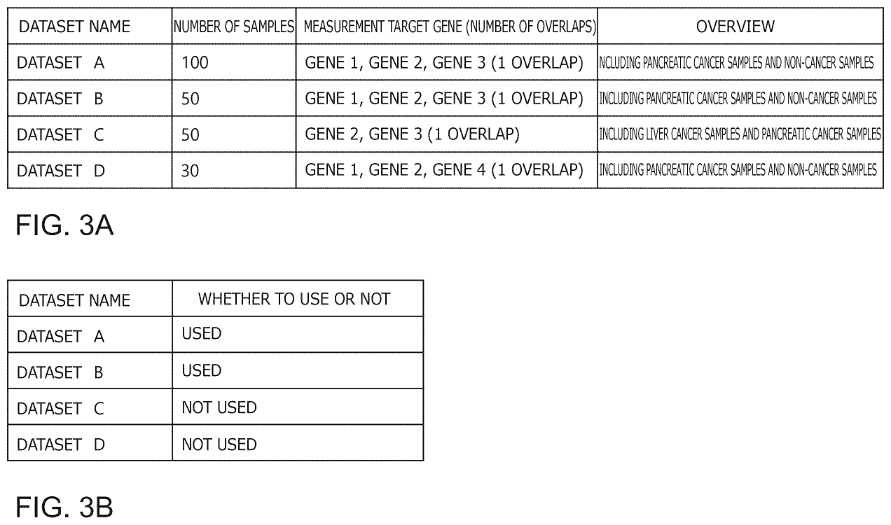

FIG. 3A is a table showing the information of the selected datasets after the duplicates of the datasets have been eliminated and datasets with the number of measured samples below the threshold have been removed, along with examples of evaluation information for each dataset. FIG. 3A shows the dataset name, the number of samples, the measurement target genes, and an overview. The number of samples and the number of overlaps are examples of the evaluation information for the dataset. The processing of selecting two or more datasets so that the number of overlaps of the measurement target genes becomes greater than or equal to the threshold has not yet been performed.

FIG. 3B is an example of dataset selection information input by the user who has visually perceived the information in FIG. 3A to narrow down the datasets to be used for the analysis. As the dataset selection information, it is instructed to use a dataset A and a dataset B and not to use a dataset C and a dataset D. Specifically, the dataset C is excluded because liver cancer that is not targeted is included therein, and the user eliminates its use. When the dataset D is excluded, the number of overlaps of measurement target genes becomes three (a gene 1, a gene 2, and a gene 3) between the dataset A and the dataset B. For this reason, the user has decided not to use the dataset C and the dataset D for analysis. This decision may be made by the dataset selector 204 instead of the user.

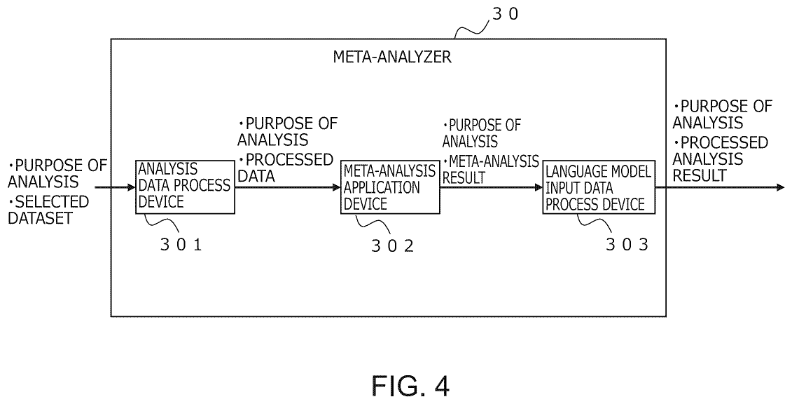

FIG. 4 is a block diagram showing an example of a configuration of the meta-analyzer 30.

The meta-analyzer 30 processes the selected datasets obtained from the dataset searcher 20 into data for analysis, applies a meta-analysis technique using the processed data, and processes the meta-analysis results, which is an analysis result data, into data for language model input to output the processed results.

The meta-analyzer 30 includes an analysis data process device 301, a meta-analysis application device 302, and a language model input data process device 303.

The analysis data process device 301 extracts necessary data or information from the selected datasets and processes the data to apply the meta-analysis to generate processed data. Specifically, data integration, data formatting, processing on missing values and outliers of data, or the like is performed. The data to be extracted may include not only the measurement values of the genes but also statistical values and effect sizes of the genes.

In this example, expression levels of microRNAs measured by a microarray are extracted, and numerical processing (logarithmic processing) is performed to ensure that scales match between datasets. Specifically, for the two datasets of the dataset A and the dataset B shown in FIG. 3B, the measurement values of the gene expression levels of the gene 1, the gene 2, and the gene 3 are extracted from MA samples included in the dataset A, and the measurement values of the gene expression levels of the gene 1, the gene 2, and the gene 3 are extracted from MB samples included in the dataset B. Logarithmic processing is performed on the data from the dataset B to ensure the scales match.

The meta-analysis application device 302 obtains the processed data from the analysis data process device 301 and applies the meta-analysis technique to generate an analysis result. The meta-analysis technique includes an integration technique such as a fixed-effect model and a random-effect model, a visualization technique such as a histogram and a forest plot, and the like. Any one of these techniques or other techniques may be used, and plural techniques may be combined. The meta-analysis application device 302 performs the data analysis by executing an analytical algorithm that realizes these techniques.

In this example, for each gene, based on a hypothesis on whether expression levels differ between the pancreatic cancer samples and the non-cancer samples, statistical values indicating differential expression in both of the samples are calculated for each dataset, and a p-value is calculated through a null hypothesis test with the statistical values integrated. Specifically, for each gene g=1 to 3, statistical values indicating differential expression in the pancreatic cancer samples and the non-cancer samples are calculated as δ_{g,A} for the dataset A and δ_{g,B} for the dataset B. The P-value P_{g} for each gene g is calculated through the null hypothesis test with the statistical values integrated.

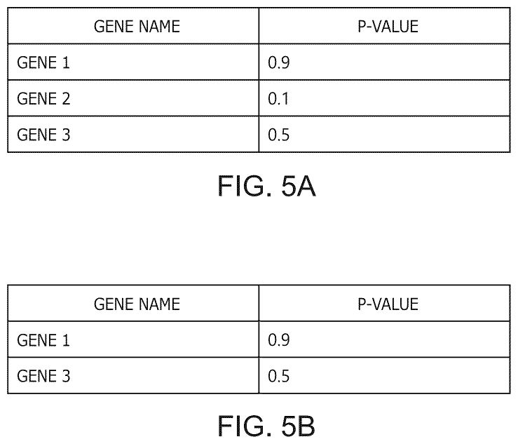

FIG. 5A is a table showing an example of the analysis results. A table including P-values for each gene is shown.

The language model input data process device 303 obtains the meta-analysis result from the meta-analysis application device 302, processes the result for language model input, and generates the processed analysis result. Specifically, key information is extracted from a table, a drawing, or the like that shows the analysis result, and the information is converted into a format in which the information can be input into a language model such as a natural language. In general, for data processing into the data for language model input, a rule-based method may be used to perform the information processing, or a statistical technique or a machine learning technique may be used to perform the information processing.

In this example, a table is generated in a text format that includes a list of microRNAs (the gene 1 and the gene 3) that are differentially expressed between the pancreatic cancer samples and the non-cancer samples, along with their P-values (P₁ and P₃).

FIG. 5B is a table showing an example of processed analysis results. A table showing the P-values of the gene 1 and the gene 3 is presented. In this example, it is not estimated that the gene 2 is differentially expressed at a significance level of α or higher because the p-value P₂ is less than the significance level α, and therefore the gene 2 is not included in the table.

The interpretation generator 40 generates an interpretation generation prompt that includes gene-related information related to the processed analysis results, based on the processed analysis results obtained from the meta-analyzer 30 and the information on the purpose of the analysis. In this case, if the user interpretation generation instruction information (described later) is obtained from the data input device 10, it is permissible to further use this interpretation generation instruction information to generate the interpretation generation prompt. The interpretation generator 40 generates and outputs interpretive information corresponding to the meta-analysis results using the generated interpretation generation prompt and the language model.

FIG. 6 is a block diagram showing a configuration example of the interpretation generator 40.

The interpretation generator 40 includes an interpretation generation prompt generator 401, a language model interpretation generator 402, a user presentation data process device 403, a gene-related information DB 404 (first database), a gene-related additional information searcher 405, and a gene-related additional information DB 406 (second database).

The gene-related information DB 404 stores or accumulates information with regard to genes. For example, the information includes information on gene sequences, functions, gene transcripts and functions thereof, relationships between genes, and the like. From the gene-related information DB 404, relevant information can be obtained for each gene.

The interpretation generation prompt generator 401 obtains processed analysis results from the meta-analyzer 30, and based on the processed analysis results and the information on the purpose of the analysis, generates an interpretation generation prompt that includes gene-related information related to the processed analysis results. The information on the purpose of the analysis may be obtained through the meta-analyzer 30 or extracted from the search instruction information inputted from the data input device 10.

The interpretation generation prompt generator 401 selects an appropriate template (template for interpretation generation prompts) according to the information on the purpose of the analysis, the processed analysis results, and the like. The template has a form adapted to a language model to be used in the language model interpretation generator 402.

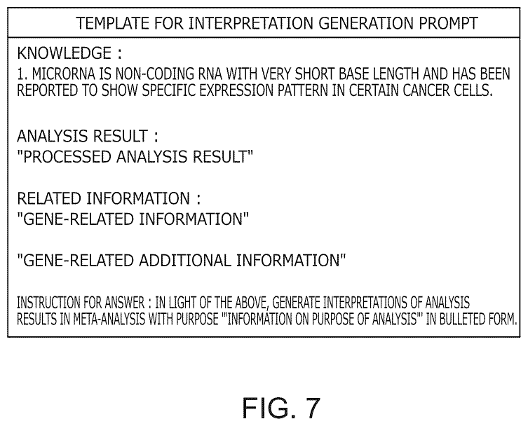

FIG. 7 shows an example of a template for interpretation generation prompts. The example in FIG. 7 is a template for the interpretation generation prompts corresponding to the meta-analysis results of microRNAs, where ‘processed analysis results’, ‘gene-related information’, ‘gene-related additional information’, and ‘information on the purpose of the analysis’ are variables. As an example, the “processed analysis results” correspond to the first variable, the “gene-related information” or the “gene-related additional information” corresponds to the second variable, and the “information on the purpose of the analysis” corresponds to the third variable. The “information on the purpose of the analysis” is included in an instruction for an answer to the language model. Texts other than variables are included in the template in advance. If there is interpretation generation instruction information from the user, different templates may be used.

The interpretation generation prompt generator 401 retrieves gene-related information related to the processed analysis results from the gene-related information DB 404.

The gene-related additional information searcher 405 searches the gene-related additional information DB 406 based on the processed analysis results from the meta-analyzer 30 to retrieve other types of gene-related information related to the processed analysis results. The gene-related additional information DB 406 stores a different type of gene-related information from that of the gene-related information DB 404. The gene-related additional information DB 406 is, for example, a DB that is updated daily. The gene-related information obtained from the gene-related additional information DB 406 is specifically referred to as gene-related additional information. The gene-related additional information searcher 405 outputs the acquired gene-related additional information to the interpretation generation prompt generator 401.

The gene-related information DB 404 and the gene-related additional information DB 406 are separate databases, but those databases may be the same database. The database storing gene-related information according to the present embodiment includes at least one of the gene-related information DB 404 and the gene-related additional information DB 406.

The interpretation generation prompt generator 401 replaces the ‘gene-related information’ and the ‘gene-related additional information’ among the variables in the template with gene-related information obtained from the gene-related information DB 404 and gene-related additional information obtained from the gene-related additional information DB 406. As a result, an intermediate interpretation generation prompt obtained by replacing some variables in the interpretation generation template is generated.

The language model interpretation generator 402 obtains an intermediate interpretation generation prompt from the interpretation generation prompt generator 401 and replaces the ‘processed analysis results’ and the ‘information on the purpose of the analysis’ among the variables in the obtained interpretation generation prompt with the processed analysis results and the information on the purpose of the analysis. As a result, the interpretation generation prompt is generated.

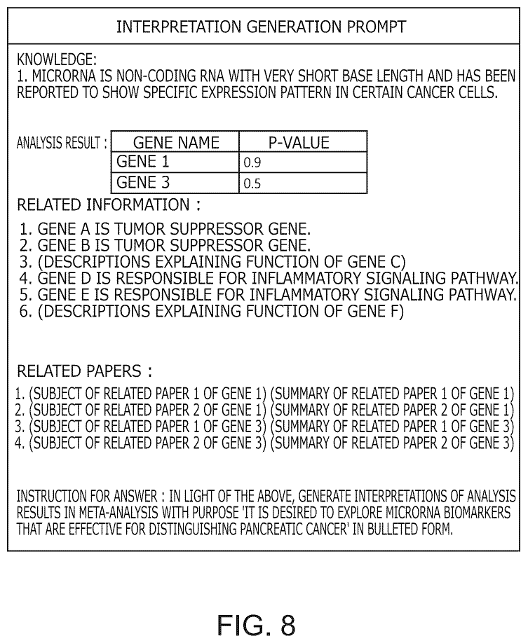

FIG. 8 shows an example of the interpretation generation prompt generated by the interpretation generation prompt generator 401. In the example of FIG. 8, prerequisite knowledge with regard to microRNAs is included in the form of a text as illustrated (note that the prerequisite knowledge is included in the template in this example). As a result of the analysis, contents of FIG. 5B are stored. Three items each of names and representative functions of biomolecules that are putative targets of the gene 1 and the gene 3 are included as the genetic-related information. In addition, two items each of subjects and summaries of papers related to the gene 1 and the gene 3 are included as other types of gene-related information. In addition, the instruction for the answer to the language model are included in the form of text as illustrated.

The language model interpretation generator 402 inputs the generated interpretation generation prompt into the language model to generate interpretive information. The interpretive information corresponds to the information on the purpose of the analysis and the processed analysis results and is the result information of the answer requested in the instruction for the answer which is included in the interpretation generation prompt. A language model is a processing device capable of understanding context and meaning from natural language sentences and generating new sentences by using a natural language processing technology. A language model may also be a large language model. However, an input and output for the language model are not limited to natural language sentences and may include images and the like.

In the present embodiment, a language model execution engine is provided in the language model interpretation generator 402, but the language model execution engine may exist as an external server and may be configured to receive interpretive information from a server by sending an interpretation generation prompt to the server. The language model may be a general-purpose language model or a language model adjusted for interpretative generation purposes.

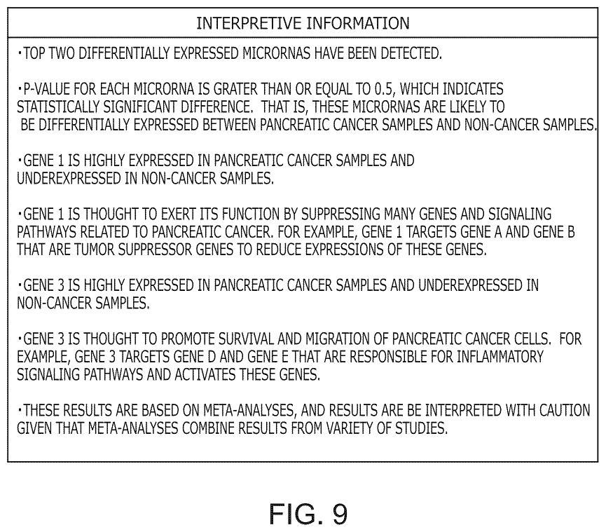

FIG. 9 shows an example of interpretive information for analysis results generated by the language model interpretation generator 402. In the example of FIG. 9, the interpretive information for the analysis results includes contents of the analysis results and considerations.

The user presentation data process device 403 evaluates a quality of interpretive information and decides whether to cause the interpretation generation prompt generator 401 to regenerate the interpretation generation prompt.

The evaluation on the quality is conducted, for example, as follows.
Whether or not the number of characters in the interpretive information is greater than or equal to a threshold is checked, and it may be determined that the amount of information is insufficient if the number of characters is less than the threshold, and a decision to regenerate the interpretation generation prompt may be made.
A numerical value to evaluate the quality of interpretive information may be generated using a language model, and the quality may be evaluated depending on whether or not the numerical value is greater than or equal to a threshold.
In addition, the quality may be evaluated using a rule-based method, or the quality may be evaluated using a statistical technique or a machine learning technique.

If it is decided to regenerate the interpretation generation prompt, the user presentation data process device 403 sends the instruction information for the regeneration of the interpretation generation prompt (interpretation generation instruction information) to the interpretation generation prompt generator 401 to obtain the interpretive information again. The interpretation generation prompt used this time can be the same as that of the last time (even the same content interpretation generation prompt may yield different answers (interpretive information)). Alternatively, the search is conducted again in the gene-related information DB 404 or the gene-related additional information DB 406 to retrieve gene-related information or gene-related additional information (for example, by increasing the number of items to be retrieved through the search as compared to the last time), and the gene-related information or gene-related additional information retrieved through the search conducted again may be additionally included in the previous interpretation generation prompt (it is not necessary to add the same information as last time). If the gene-related information DB 404 or the gene-related additional information DB 406 is updated over time, it is possible to retrieve gene-related information or gene-related additional information that has not been found in the previous search. Alternatively, new information (additional information) may be added to the previous interpretation generation prompt. The additional information may be generated in accordance with a user's input of interpretation generation instruction information which will be described later. The generation of interpretive information is repeated until the quality meets a termination condition (for example, exceeding a threshold or reaching a maximum number of repetitions).

The user presentation data process device 403 processes, based on the information on the purpose of the analysis, the data into a format in which processed analysis results and interpretive information can be presented to the user to obtain user presentation data. For example, data that includes the processed analysis results from FIG. 5B and the interpretive information from FIG. 9 is generated as the user presentation data. The user presentation data may be presented to the user via the data output device 50. Other information such as information on the purpose of the analysis may be included in the user presentation data presented to the user.

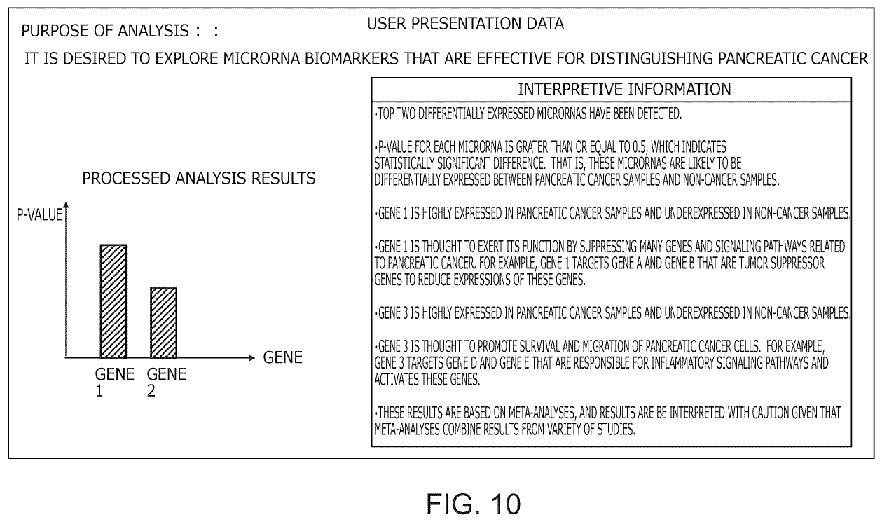

FIG. 10 shows an example of the user presentation data presented to the user. The information on the purpose of the analysis, graph data of the processed analysis results, and the interpretive information are presented.

The user may input interpretation generation instruction information from data input device 10 in a case where the user is not satisfied with the interpretive information by checking the user presentation data or the like, and the user may cause the language model interpretation generator 402 to generate interpretive information again. In this case, the interpretation generation prompt generator 401 regenerates the interpretation generation prompt based on the user's interpretation generation instruction information and sends the interpretation generation prompt to the language model interpretation generator 402 to cause the language model interpretation generator 402 to generate the interpretive information again.

The interpretation generation instruction information from the user may be, for example, contents for an instruction to additionally include the interpretation generation results of the same content queries conducted in the past, for example, in the interpretation generation prompt generated immediately before. As a result, it can also be expected to obtain a comment on a difference between this consideration (interpretation result) and the previous consideration (interpretation result) in the regenerated interpretive information, for example. If the user has additional knowledge or a question regarding the considerations included in the interpretive information, the interpretive information may be regenerated by additionally including the additional knowledge or question in the interpretation generation prompt. This enables the generation of dialog-style interpretations. The user may also instruct to search the gene-related information DB 404 or gene-related additional information DB 406 again based on the interpretation generation instruction information to retrieve gene-related information or gene-related additional information (for example, increasing the number of items to be retrieved through the search as compared to the last time) and additionally include the gene-related information or gene-related additional information retrieved through the search conducted again in the previous interpretation generation prompt (it is not necessary to add the same information as the last time). If the gene-related information DB 404 or the gene-related additional information DB 406 is updated over time, gene-related information or gene-related additional information that has not been found in the previous search may be retrieved.

The data output device 50 outputs the information of the selected datasets (see FIG. 3A), the dataset evaluation results (refer to the “number of overlaps” in FIG. 3A and “not used”, “used,” and the like in FIG. 3B), the processed analysis results (see FIG. 5B), the interpretive information (see FIG. 9), the user presentation data, and the like.

The configuration in which the data output device 50 outputs these information or data may be optional as long as the configuration allows the user to recognize the information or data. For example, the data output device 50 may be a display such as a data liquid crystal display device or an organic EL display device and may display information or data using visual elements such as images or natural language. The data output device 50 may be a printer and may print information or data using visual elements such as images and natural language. The data output device 50 may be an application and may save information or data to a file in an appropriate file format. The data output device 50 may be a communication apparatus capable of transmitting and receiving information or data with a user's terminal or server and may transmit information or data via a network.

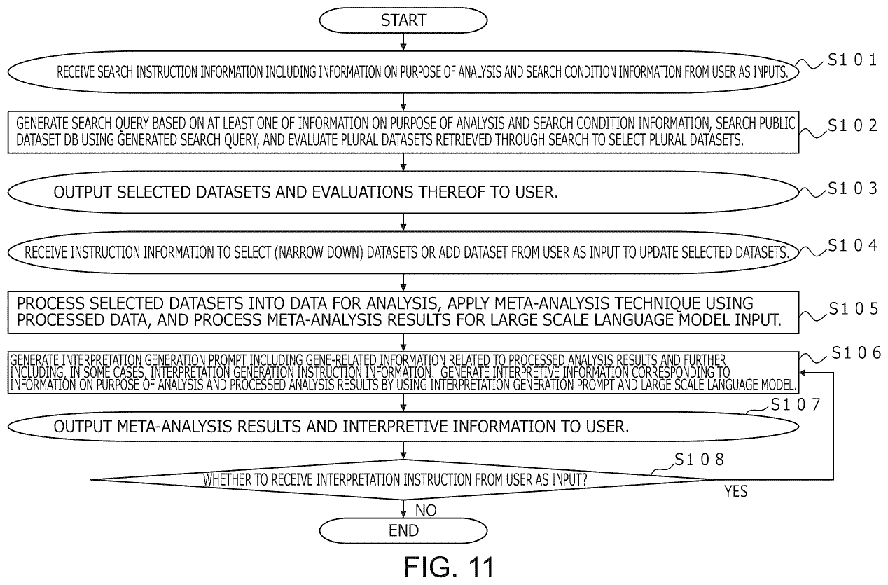

FIG. 11 is a flowchart schematically showing a procedure in the information processing apparatus according to Embodiment 1 of the present invention. The following is one example, and it is not necessary to conduct dataset searches or meta-analyses or to generate interpretive information exactly as described.

In step S101, the input device 10 receives search instruction information as an input from the user. The search instruction information includes, as an example, information on the purpose of the analysis and search condition information.

In step S102, the dataset searcher 20 generates a search query based on at least one of the information on the purpose of the analysis and the search condition information and searches the public dataset DB 205 using the generated search query. The dataset evaluator 203 evaluates plural datasets retrieved through the search and selects plural datasets based on the evaluation results.

In step S103, the output device 50 outputs information of the selected datasets and information of the evaluation results to the user (see FIG. 3A).

In step S104, the input device 10 receives information (database selection information) for an instruction to select (narrow down) the datasets or information (database addition instruction selected information) for an instruction to add a database from the user. The database selection information is the instruction information entered when the user desires to further narrow down the selected datasets that have been output. The database addition instruction information is the instruction information entered when the user desires to further add a dataset. The dataset selector 204 updates the set of selected datasets based on one or both of the database selection information and the database addition instruction information. When the database selection information is entered, the number of selected datasets decreases, and when the database addition instruction information is entered, the number of selected datasets increases. There may be cases where the user does not enter any instruction information.

In step S105, the meta-analyzer 30 processes the selected datasets for analysis, applies the meta-analysis technique to the processed data to obtain the meta-analysis results, and processes the meta-analysis results for language model input. For example, the necessary information is extracted from the selected dataset information and the data is processed to apply the meta-analysis to generate the processed data. For example, the expression levels of microRNAs are extracted to perform numerical processing (logarithmic processing) so as to ensure that the scales match between datasets. For each gene, based on a hypothesis on whether expression levels differ between the pancreatic cancer samples and the non-cancer samples, statistical values indicating differential expression in both of the samples are calculated for each dataset, and a p-value is calculated through a null hypothesis test with the statistical values integrated (see FIG. 5A). A table is generated in a text format that includes a list of genes that are differentially expressed between the pancreatic cancer samples and the non-cancer samples, along with their P-values (see FIG. 5B).

In step S106, the interpretation generator 40 generates an interpretation generation prompt that includes gene-related information related to the processed analysis results, and others. If there is an input of interpretation generation instruction information, the information specified by the interpretation generation instruction information is also included in the interpretation generation prompt (see FIG. 8). By using an interpretation generation prompt and a language model, interpretive information (see FIG. 9) corresponding to the information on the purpose of the analysis and the processed analysis results is generated.

In step S107, the output device 50 outputs the processed analysis results (see FIG. 5B) and the interpretive information (see FIG. 9) to the user.

In step S108, if the data input device 10 receives the interpretation generation instruction information from the user as an input, the flow returns to step S106, and if the interpretation generation instruction information is not received, the present processing ends.

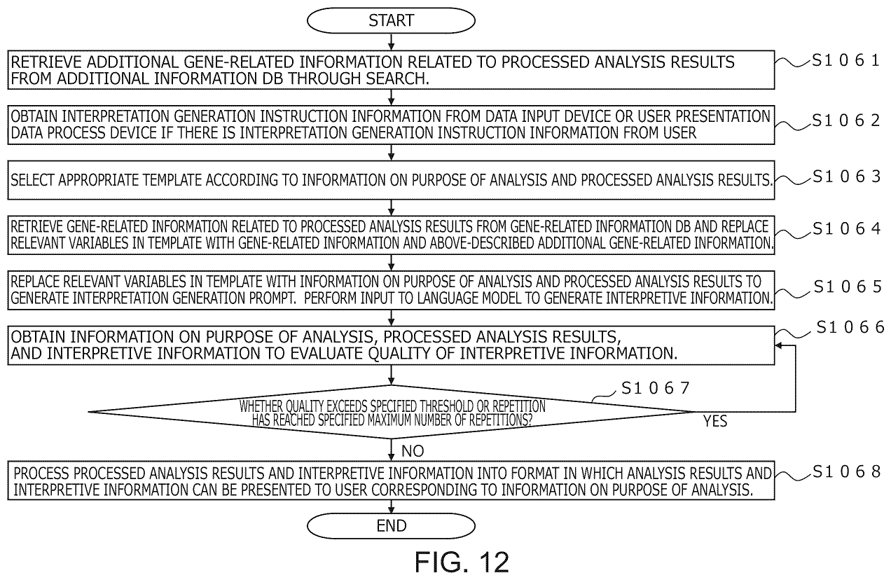

FIG. 12 is a flowchart showing an example of the detailed operation of step S106 in FIG. 11. This flowchart provides a more detailed example of the procedure for generating interpretive information for analysis results.

In step S1061, the gene-related additional information searcher 405 retrieves other types of gene-related information (additional gene-related information) related to the processed analysis results by searching the gene-related additional information DB 406. For example, a search is performed in papers with regard to the genes (the gene 1 and the gene 3) included in the processed analysis results to retrieve two subjects and summaries for each.

In step S1062, if there is interpretation generation instruction information from the user or interpretation generation instruction information from the user presentation data process device 403, the interpretation generation prompt generator 401 obtains the interpretation generation instruction information from the data input device 10 or the user presentation data process device 403.

In step S1063, the interpretation generation prompt generator 401 selects an appropriate template according to at least one of the information on the purpose of the analysis and the processed analysis results.

In step S1064, the interpretation generation prompt generator 401 retrieves gene-related information related to the processed analysis results from the gene-related information DB 404. The retrieved gene-related information and the additional gene-related information retrieved in step S1061 are added to the template. Specifically, relevant variables in the selected template are replaced with the retrieved gene-related information and the additional gene-related information retrieved in step S1061. If interpretation generation instruction information has been obtained, the contents indicated by the interpretation generation instruction information may be further added into the template. By adding the contents instructed by the interpretation generation instruction information, it is also possible to generate interpretations in a dialogue format.

In step S1065, the information on the purpose of the analysis and the processed analysis results are added to the relevant sections in the template. Specifically, the relevant variables in the template are replaced with the information on the purpose of the analysis and the processed analysis results. In steps S1064 and S1065, all variables in the template are replaced (information necessary for interpretation generation is added to the template), resulting in an interpretation generation prompt. In addition, part of the processing of generating the interpretation generation prompt may be performed by the language model interpretation generator 402. For example, the processing of adding the information on the purpose of the analysis and the processed analysis results to the template may be performed by the language model interpretation generator 402.

In step S1066, the user presentation data process device 403 obtains interpretive information from the language model interpretation generator 402 and evaluates the quality of the interpretive information based on the information on the purpose of the analysis and the processed analysis results. For example, the evaluation may be performed based on the number of characters in the interpretive information, or a numerical value may be generated to evaluate the quality of the interpretive information using a language model. In addition, the quality may be evaluated using the rule-based method, or the quality may be evaluated using the statistical technique or the machine learning technique.

In step S1067, if the quality exceeds a specified threshold or the repetition has reached a specified maximum number of repetitions, the flow proceeds to step S1068. If not, interpretation generation instruction information to instruct the regeneration of the interpretation generation prompt is generated by the user presentation data process device 403, and the flow return to step S1062.

In step S1068, the user presentation data process device 403 processes, based on the information on the purpose of the analysis, the data into a format in which the processed analysis results and interpretive information can be presented to the user, and presents the processed results to the user via the data output device 50.

As described above, according to the present embodiment, it is possible to automate the meta-analysis without requiring advanced expertise in genetic information, enabling analysis to be conducted in a short period.

### (Modified Example 1)

The information on the purpose of the analysis may include “microRNAs related to large intestine cancer in Japanese people”, “DNA sequences related to type 2 diabetes in Japanese people”, “RNA sequences related to cerebral ischemic diseases”, “mRNA sequences related to mental disorders”, and the like. The search condition information may include information indicating conditions such as using databases like “ENA database” or “DDBJ database” in the search database, or information indicating conditions such as using measurement instrument like “PCR” or “NGS”.

### (Modified Example 2)

The language model interpretation generator 402 may generate data process instruction information in addition to interpretive information, and the user presentation data process device 403 may process data such as processed analysis results according to the data process instruction information to generate additional interpretive information and output the additional interpretive information together with the interpretive information. For example, the language model interpretation generator 402 processes, as interpretive information, data such as processed analysis results in addition to a document representing interpretive information to generate instruction information (data process instruction information) for generating a graph that indicates the information. The user presentation data process device 403 can generate a graph according to the data process instruction information and output user presentation data in which the document representing the interpretive information and the graph are combined.

### (Modified Example 3)

In the template of FIG. 7, the knowledge section includes information (“1. MicroRNA is a non-coding RNA with a very short base length and has been reported to show a specific expression pattern in certain cancer cells.”) in advance, but it may also be possible to treat knowledge as a variable and embed information into the template. In this case, for example, the gene-related information DB 404 may be accessed based on the information included in the query that has been input by the user to retrieve related information corresponding to the information to embed the related information in the knowledge section of the template. For example, if the query includes “microRNA”, the gene-related information DB 404 may be accessed about the microRNA, and the relevant related information may be retrieved to embed the information in the knowledge section of the template.

### (Hardware Configuration)

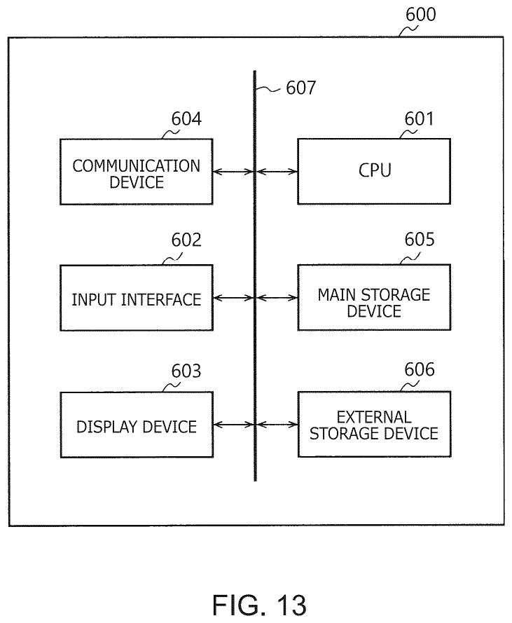

FIG. 13 illustrates a hardware configuration of the information processing apparatus 100 according to each embodiment. The information processing apparatus 100 is configured as a computer device 600. The computer device 600 includes a CPU 601, an input interface 602, a display device 603, a communication device 604, a main storage device 605, and an external storage device 606, and these components are mutually connected through a bus 607.

The CPU (central processing unit) 601 executes an information processing program as a computer program on the main storage device 605. The information processing program is a computer program configured to achieve each above-described functional composition of the present device. The information processing program may be achieved by a combination of a plurality of computer programs and scripts instead of one computer program. Each functional composition is achieved as the CPU 601 executes the information processing program.

The input interface 602 is a circuit for inputting, to the present device, an operation signal from an input device such as a keyboard, a mouse, or a touch panel. The input interface 602 corresponds to the data input device 10.

The display device 603 displays data output from the present device. The display device 603 is, for example, a liquid crystal display (LCD), an organic electroluminescence display, a cathode-ray tube (CRT), or a plasma display (PDP) but is not limited thereto. Data output from the computer device 600 can be displayed on the display device 603. The display device 603 corresponds to the data output device 50.

The communication device 604 is a circuit for the present device to communicate with an external device in a wireless or wired manner. Data can be input from the external device through the communication device 604. The data input from the external device can be stored in the main storage device 605 or the external storage device 606. The communication device 604 corresponds to the data output device 50.

The main storage device 605 stores, for example, the information processing program, data necessary for execution of the information processing program, and data generated through execution of the information processing program. The information processing program is loaded and executed on the main storage device 605. The main storage device 605 is, for example, a RAM, a DRAM, or an SRAM but is not limited thereto. Each storage or database in the information processing apparatus in each embodiment may be implemented on the main storage device 605.

The external storage device 606 stores, for example, the information processing program, data necessary for execution of the information processing program, and data generated through execution of the information processing program. The information processing program and the data are read onto the main storage device 605 at execution of the information processing program. The external storage device 606 is, for example, a hard disk, an optical disk, a flash memory, or a magnetic tape but is not limited thereto. Each storage or database in the information processing apparatus in each embodiment may be implemented on the external storage device 606.

The information processing program may be installed on the computer device 600 in advance or may be stored in a storage medium such as a CD-ROM. Moreover, the information processing program in each embodiment may be uploaded on the Internet.

The information processing apparatus 100 may be configured as a single computer device 600 or may be configured as a system including a plurality of mutually connected computer devices 600.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

The embodiments of the present invention can also be configured as follows.

### CLAUSES

Clause 1. An information processing apparatus comprising processing circuitry configured to:
   analyze data regarding genetic information according to a purpose of a data analysis and obtain analysis result data; and
   generate a prompt for inputting into a language model, the prompt instructing to interpret the analysis result data based on gene-related information related to the analysis result data and the purpose of the data analysis and obtain an interpretation result of the analysis result data based on the prompt and the language model.
Clause 2. The information processing apparatus according to Clause 1, wherein the processing circuitry obtains a template for creating the prompt, the template including a first variable for storing the analysis result data, a second variable for storing the gene-related information related to the analysis result data, and a third variable for storing the purpose of the data analysis, and
   stores the analysis result data in the first variable of the template, stores the genetic information in the second variable, and stores the purpose of the data analysis in the third variable to generate the prompt.
Clause 3. The information processing apparatus according to Clause 1 or 2, wherein the processing circuitry evaluates a quality of the interpretation result, regenerates the prompt according to an evaluation result, and obtains an interpretation result of the analysis result data based on the regenerated prompt and the language model.
Clause 4. The information processing apparatus according to Clause 3, comprising:
   an output device configured to output the interpretation result; and
   an input device configured to receive information as an input, wherein
   the processing circuitry obtains additional information with respect to the prompt from the input device and regenerates the prompt further based on the obtained additional information.
Clause 5. The information processing apparatus according to Clause 4, wherein
   the obtained additional information includes a user's question with respect to the interpretation result, and
   the processing circuitry obtains the interpretation result of the analysis result data and an answer to the question based on the regenerated prompt and the language model.
Clause 6. The information processing apparatus according to Clause 4 or 5, wherein
   the obtained additional information includes an interpretation result obtained in a past for a data analysis with a purpose same as the purpose of the data analysis, and
   the processing circuitry obtains information explaining a difference between the interpretation result of the analysis result data and the interpretation result obtained in the past based on the regenerated prompt and the language model.
Clause 7. The information processing apparatus according to any one of Clauses 1 to 6, wherein the processing circuitry searches a first database that stores plural kinds of gene-related information based on the analysis result data, and retrieves the gene-related information related to the analysis result data.
Clause 8. The information processing apparatus according to Clause 7, wherein
   the processing circuitry searches a second database that stores plural other kinds of gene-related information different from the gene-related information based on the analysis result data, and retrieves other gene-related information related to the analysis result data, and
   generates the prompt further based on the obtained other gene-related information.
Clause 9. The information processing apparatus according to any one of Clauses 1 to 8, wherein the analysis result data includes plural genes according to the purpose of the data analysis.
Clause 10. The information processing apparatus according to Clause 9, wherein the gene-related information includes information of genes that are putative targets for the genes included in the analysis result data.
Clause 11. The information processing apparatus according to Clause 10, wherein
   the processing circuitry generates the prompt further based on other gene-related information which is different from the gene-related information and which is related to the analysis result data, and
   the other gene-related information is information of papers related to the genes included in the analysis result data.
Clause 12. The information processing apparatus according to any one of Clauses 1 to 11, wherein the processing circuitry performs the data analysis by executing an analysis algorithm according to the purpose of the data analysis.
Clause 13. The information processing apparatus according to Clause 12, wherein the data analysis is a meta-analysis.
Clause 14. The information processing apparatus according to any one of Clauses 1 to 13, wherein the data related to the genetic information is a dataset that includes measurement data regarding genes for plural samples.
Clause 15. The information processing apparatus according to any one of Clauses 1 to 14, wherein the processing circuitry searches a third database that stores plural data related to genetic information based on at least one of the purpose of the data analysis and a search condition of the data related to the genetic information, and retrieves the data related to the genetic information.
Clause 16. The information processing apparatus according to Clause 15, wherein
   the search condition includes at least one of a condition that specifies a database to be used for the search and a condition that specifies measuring instrument used to measure the genetic information, and
   the processing circuitry searches a database that meets the search condition or searches for data in which the genetic information is measured by the measuring instrument.
Clause 17. The information processing apparatus according to Clause 15 or 16, wherein
   the data related to the genetic information and each of the data in the third database are a dataset that includes measurement data regarding genes for plural samples, and
   the processing circuitry retrieves plural datasets from the third database, and
   selects, based on a number of samples in each of the obtained plural datasets and measurement target genes in each of the obtained plural datasets, some of the obtained plural datasets, and analyzes the selected plural datasets to obtain the analysis result data.
Clause 18. The information processing apparatus according to Clause 17, comprising:
   an output device configured to output information from the selected plural datasets; and
   an input device configured to receive information as an input, wherein
   the processing circuitry obtains, from the input device, selection information that instructs selection of a part of the selected plural data sets or addition instruction information that instructs addition of a new data set, and
   updates a set of the selected plural datasets based on the selection information or the addition instruction information and analyzes the datasets in the updated set to obtain the analysis result data.
Clause 19. An information processing method comprising:
   analyzing data regarding genetic information according to a purpose of a data analysis and obtain analysis result data; and
   generating a prompt for inputting into a language model, the prompt instructing to interpret the analysis result data based on gene-related information related to the analysis result data and the purpose of the data analysis and obtaining an interpretation result of the analysis result data based on the prompt and the language model.
Clause 20. A non-transitory computer readable medium having a computer program stored therein which causes a computer to perform processes comprising:
   analyzing data regarding genetic information according to a purpose of a data analysis and obtain analysis result data; and
   generating a prompt for inputting into a language model, the prompt instructing to interpret the analysis result data based on gene-related information related to the analysis result data and the purpose of the data analysis and obtaining an interpretation result of the analysis result data based on the prompt and the language model.

1. An information processing apparatus comprising processing circuitry configured to:
analyze data regarding genetic information according to a purpose of a data analysis and obtain analysis result data; and
generate a prompt for inputting into a language model, the prompt instructing to interpret the analysis result data based on gene-related information related to the analysis result data and the purpose of the data analysis and obtain an interpretation result of the analysis result data based on the prompt and the language model.

2. The information processing apparatus according to claim 1, wherein the processing circuitry obtains a template for creating the prompt, the template including a first variable for storing the analysis result data, a second variable for storing the gene-related information related to the analysis result data, and a third variable for storing the purpose of the data analysis, and
stores the analysis result data in the first variable of the template, stores the genetic information in the second variable, and stores the purpose of the data analysis in the third variable to generate the prompt.

3. The information processing apparatus according to claim 1, wherein the processing circuitry evaluates a quality of the interpretation result, regenerates the prompt according to an evaluation result, and obtains an interpretation result of the analysis result data based on the regenerated prompt and the language model.

4. The information processing apparatus according to claim 3, comprising:
an output device configured to output the interpretation result; and
an input device configured to receive information as an input, wherein
the processing circuitry obtains additional information with respect to the prompt from the input device and regenerates the prompt further based on the obtained additional information.

5. The information processing apparatus according to claim 4, wherein
the obtained additional information includes a user's question with respect to the interpretation result, and
the processing circuitry obtains the interpretation result of the analysis result data and an answer to the question based on the regenerated prompt and the language model.

6. The information processing apparatus according to claim 4, wherein
the obtained additional information includes an interpretation result obtained in a past for a data analysis with a purpose same as the purpose of the data analysis, and
the processing circuitry obtains information explaining a difference between the interpretation result of the analysis result data and the interpretation result obtained in the past based on the regenerated prompt and the language model.

7. The information processing apparatus according to claim 1, wherein the processing circuitry searches a first database that stores plural kinds of gene-related information based on the analysis result data, and retrieves the gene-related information related to the analysis result data.

8. The information processing apparatus according to claim 7, wherein
the processing circuitry searches a second database that stores plural other kinds of gene-related information different from the gene-related information based on the analysis result data, and retrieves other gene-related information related to the analysis result data, and
generates the prompt further based on the obtained other gene-related information.

9. The information processing apparatus according to claim 1, wherein the analysis result data includes plural genes according to the purpose of the data analysis.

10. The information processing apparatus according to claim 9, wherein the gene-related information includes information of genes that are putative targets for the genes included in the analysis result data.

11. The information processing apparatus according to claim 10, wherein
the processing circuitry generates the prompt further based on other gene-related information which is different from the gene-related information and which is related to the analysis result data, and
the other gene-related information is information of papers related to the genes included in the analysis result data.

12. The information processing apparatus according to claim 1, wherein the processing circuitry performs the data analysis by executing an analysis algorithm according to the purpose of the data analysis.

13. The information processing apparatus according to claim 12, wherein the data analysis is a meta-analysis.

14. The information processing apparatus according to claim 1, wherein the data related to the genetic information is a dataset that includes measurement data regarding genes for plural samples.

15. The information processing apparatus according to claim 1, wherein the processing circuitry searches a third database that stores plural data related to genetic information based on at least one of the purpose of the data analysis and a search condition of the data related to the genetic information, and retrieves the data related to the genetic information.

16. The information processing apparatus according to claim 15, wherein
the search condition includes at least one of a condition that specifies a database to be used for the search and a condition that specifies measuring instrument used to measure the genetic information, and
the processing circuitry searches a database that meets the search condition or searches for data in which the genetic information is measured by the measuring instrument.

17. The information processing apparatus according to claim 15, wherein
the data related to the genetic information and each of the data in the third database are a dataset that includes measurement data regarding genes for plural samples, and
the processing circuitry retrieves plural datasets from the third database, and
selects, based on a number of samples in each of the obtained plural datasets and measurement target genes in each of the obtained plural datasets, some of the obtained plural datasets, and analyzes the selected plural datasets to obtain the analysis result data.

18. The information processing apparatus according to claim 17, comprising:
an output device configured to output information from the selected plural datasets; and
an input device configured to receive information as an input, wherein
the processing circuitry obtains, from the input device, selection information that instructs selection of a part of the selected plural data sets or addition instruction information that instructs addition of a new data set, and
updates a set of the selected plural datasets based on the selection information or the addition instruction information and analyzes the datasets in the updated set to obtain the analysis result data.

19. An information processing method comprising:
analyzing data regarding genetic information according to a purpose of a data analysis and obtain analysis result data; and
generating a prompt for inputting into a language model, the prompt instructing to interpret the analysis result data based on gene-related information related to the analysis result data and the purpose of the data analysis and obtaining an interpretation result of the analysis result data based on the prompt and the language model.

20. A non-transitory computer readable medium having a computer program stored therein which causes a computer to perform processes comprising:
analyzing data regarding genetic information according to a purpose of a data analysis and obtain analysis result data; and
generating a prompt for inputting into a language model, the prompt instructing to interpret the analysis result data based on gene-related information related to the analysis result data and the purpose of the data analysis and obtaining an interpretation result of the analysis result data based on the prompt and the language model.
